(19) United States
(12) Patent Application Publication
(10) Pub. No.: US 2004/0072789 A1
(43) Pub. Date: Apr. 15, 2004

(54) Veterinary composition for the topical treatment of traumatized or inflamed skin

(76) Inventors: Kenneth James, Wantage (GB); Frederick James Smith, Abingdon (GB)

Correspondence Address: JACOBSON HOLMAN PLLC, 400 SEVENTH STREET N.W., SUITE 600, WASHINGTON, DC, 20004, US

(21) Appl. No.: 10/415,341

(22) PCT Filed: Nov. 5, 2001
(86) PCT No.: PCT/GB01/04881

(30) Foreign Application Priority Data
Nov. 3, 2000 (GB) 0026923.3

(51) Int. Cl. A61K 31/7024

(52) U.S. Cl. 514/53

(57) ABSTRACT

The present invention relates to a vetinary medicament comprising a sucrose ester or mixture of sucrose esters and the use of said vetinary composition in the treatment or prophylaxis of traumatized or inflamed tissue. In particular, the present invention relates to the treatment of traumatized or inflamed skin of animals including horses and cattle, said traumatized or inflamed skin resulting from, for example, microbial or fungal infections, or from insect bites.

The present invention relates to a veterinary composition for the topical treatment or prophylaxis of traumatized or inflamed tissue surfaces, for example skin. In particular, for the treatment of traumatized or inflamed tissue surfaces resulting from microbial and fungal infections, such as mud fever and rain scald, which occur on horses and ponies, and ringworm, which occurs on horses, cattle and other ruminants. Further, for the treatment of insect bites and stings, such as wasp stings and conditions such as sweet itch on horses caused by insect bites.

Mud fever affects horses and ponies, and is not a single disease, but a collection of clinical signs associated with a variety of causes. It can range from a mild skin irritation to very painful sores with an underlying infection, and can affect the whole of the horse, being known as “rain rash” or “rain scald” when affecting the back of a horse. Mud fever generally affects the horses' legs causing inflammation of the skin, which at best is uncomfortable and at worst can cause severe pain, bleeding and lameness. There are believed to be three factors which cause the condition. 1) Predisposing factors, which include the horse's genetic makeup. Any horse with white skin on the lower limbs or a long, shaggy coat around the fetlocks and feet is prone to mud fever. The animal's environment is also important, since anything that irritates, softens or breaks the skin so that infection can enter, such as cold, wet and muddy conditions, makes the mud fever syndrome more likely. 2) Primary factors, which include physical and chemical irritants, such as contact allergies and badly rubbing boots. Also, infestations of tiny mites (chorioptic mange mites) and fungal infections can be responsible for skin damage. 3) Perpetuating factors, which take advantage of the skin being compromised by the two factors above, allowing the disease to persist and progress. For example, infections by bacteria, such as Staphyloccocus and Dermatophilus.

A further development of the mud fever syndrome is a condition known as “cracked heels”. This can occur in severe cases of mud fever when the skin at the back of the leg splits open producing deep, horizontal cracks.

The treatment or prophylaxis of conditions like mud fever has proved difficult, and most prior art references to the subject teach prevention, rather than cure. Attempts to treat mud fever have been by the use of ointments to soothe and maintain hydration of the skin and in persistent cases, by the controlled use of antibiotics and anti-inflammatory drugs. However, continued use of both antibiotics and anti-inflammatory drugs is extremely undesirable and expensive. Furthermore, mite infestations and fungal infections are not sensitive to antibiotics, and such treatments therefore frequently do not cure the conditions.

Additionally, medicaments such as “griseofulvin” produced by Glaxo Laboratories are known, this being an antibiotic for the treatment of fungal infections to the hair and skin, such as ringworm. However, these are specifically taken internally in the form of tablets, by injection or a drench, enabling the medicament to be delivered to the treatment site internally via the blood supply. Topical administration of this and other similar medicaments has been attempted, but has been found to have a very limited effect on fungal infections of the skin, leading away from the topical application of medicaments when treating fungal and microbial infections of the skin.

Sweet itch is a further condition that affects horses, causing acute itching leading to traumatized skin. Sweet itch is caused by insect bites, particularly by Culicoides midges. Typically, hypersensitivity or an allergic reaction occurs in some horses as a response to midge saliva, which gets into the skin when the midge feeds. Other insects, such as black flies (Simuliidae) and stable flies (Stomoxys calcitrans) can cause a similar itchy skin disease.

Conventional treatments for conditions such as sweet itch, include the use of steroids, either by injection or by mouth. However, continued high use of steroids is both extremely undesirable for the animal, and expensive. Antihistamines and oil supplements, such as evening primrose oil, cold marine fish oil and aloe vera, are of limited benefit.

GB 2225942 discloses the use of a topical medicine containing calcium soap, such as calcium stearate, to prevent or treat inflammatory skin disease in horses and ponies. However, it is believed that such compositions are of limited benefit.

It is an object of the present invention to provide a veterinary composition for the topical treatment or prophylaxis of traumatized or inflamed tissue surfaces, such as skin, resulting from microbial and fungal infections, such as mud fever, rain scald and ringworm.

It is a further object of the present invention to provide a veterinary composition for the topical treatment or prophylaxis of traumatized or inflamed tissue surfaces resulting from insect bites and stings.

According to a first aspect of the present invention there is provided the use of a veterinary composition comprising a sucrose ester or a mixture of sucrose esters for the manufacture of a medicament for the topical treatment or prophylaxis of traumatized or inflamed tissue surfaces.

Preferably, the veterinary composition comprises a sucrose ester or a mixture of sucrose esters and calcium or zinc stearate in a veterinarially acceptable carrier. The ratio of sucrose esters to calcium stearate may be between 2:1 to 6:1.

Again preferably, the sucrose esters comprise a mixture of sucrose monopalmitate and sucrose dipalmitate.

The use of the veterinary composition may be for the manufacture of a medicament for the topical treatment or prophylaxis of traumatized or inflamed tissue surfaces resulting from fungal or microbial infections, such as mud fever, rain scald and ringworm.

Alternatively, the veterinary composition may be for the manufacture of a medicament for the topical treatment or prophylaxis of traumatized or inflamed tissue surfaces resulting from insect bites and stings, such as, sweet itch. The veterinary composition may have an analgesic effect.

Conventionally, sucrose esters are used as dispersing and emulsifying agents in food and cosmetic preparations.

Additionally, sucrose esters are known to be useful in increasing the, shelf life of fruit produce, since application of sucrose esters produces a membrane which traps a layer of water and is selectively permeable to gases, allowing carbon dioxide to be released, but preventing entry of oxygen. Accordingly, it is believed that prevention of the entry of oxygen from the air to a traumatized site on the skin of a human or animal would cause the body to deliver extra blood oxygen to the site, increasing the body's rate of healing.

Further examples of known uses of sucrose esters are disclosed in the following documents:

EP 0225367 B1 discloses a film forming composition for topical use on both humans and animals, for the treatment of skin ailments, such as acne and also for injuries where the skin has been damaged, such as burns. The composition comprises water and a film forming component at least partially soluble or dispersible in said water, the film forming component comprising a sucrose fatty acid ester or mixture of sucrose fatty acid esters, wherein the quantity of sucrose ester or sucrose esters is 40% to 80% (w/w) of the composition excluding water. However, EP 0225367 B1 does not consider the specific use of sucrose esters in a veterinary composition for the topical treatment of traumatized or inflamed skin, in particular, resulting from fungal or microbial infections, such as mud fever, rain scald and ringworm, or resulting from insect bites and stings, such as sweet itch. Moreover, EP 0225367 B1 does not mention or recognise the analgesic effect that the veterinary composition according to the present invention has been found to have.

It has been found that three main factors are required for rapid wound healing; the first is a source of calcium ions; the second is mixed glycerides; and the third is a supply of carbohydrates. In this regard, the veterinary composition according to the present invention clearly contains all three elements required for rapid wound healing. Calcium stearate is known to supply a source of calcium ions and sucrose esters providing a source of carbohydrates, along with other benefits, such as anti-fungal properties and associated analgesic effects. Prior art disclosures have been found to be less effective in the healing properties provided, for example, the composition disclosed in EP 0225367 does not include a source of calcium ions.

EP 0343671 A2 discloses a pharmaceutical composition for the external treatment of skin wounds, comprising 2 to 40% of the total weight of the composition of a mono- or disaccharide fatty acid ester and a prophylactively effective amount of an anti-infective means. However, the veterinary composition according to the present invention does not include anti-infective means.

U.S. Pat. No. 3,896,238, U.S. Pat. No. 4,150,114 and U.S. Pat. No. 4,046,886 disclose the use of a sucrose ester in combination with an alkyl sulfoxide or phosphine oxide in compositions for enhancing the penetration of pharmacologically active agents into the skin.

EP 0305493 B1 discloses a cosmetic base composition that exhibits utility as a pharmaceutical compound comprising 1% to 15% by weight sucrose fatty acid ester, 3% to 45% by weight acyl fatty acid lactylate ester or alkali metal salt thereof, and a solvent.

The sucrose ester or mixture of sucrose esters is the principal active component in the veterinary composition according to the present invention. The percentage of sucrose ester in the veterinary composition according to the present invention is between 1-50%, with the preferred percentage being 10-20% and more exactly 15%. Sucrose esters in the range C₁₂-C₂₂ are preferably employed, and saturated esters may be substituted for unsaturated esters. Preferably, a mixture of sucrose monopalmitate and sucrose dipalmitate is used. Such sucrose fatty acid esters are commonly used in foods and drug products and, accordingly, are easily available from commercial sources.

Calcium stearate acts as a source of Ca²⁺ ions. A ratio of sucrose ester to calcium stearate of 2:1 to 6:1 may be used, preferably 3.75:1.

The composition of the present invention may be in the form of a solution, suspension or emulsion, but for application to skin it is preferably in the form of an emulsion. Accordingly, it is desirable to add one or more emulsifying agents. Solvents for use in the composition of the present invention may include water, glycerol, cetyl alcohol or any other suitable solvent.

The composition of the present invention may include further ingredients to improve its properties or ease of application. An additive which improves the “feel” of the composition when applied to the tissue or skin may also be included. One such additive is a mixture of mono and diglycerides although other materials could be employed for this purpose, such as lanolin, jojoba oil, coconut oil and similar materials. The composition of the present invention may also include various other agents and ingredients commonly employed in dermatological ointments and lotions, for example, preservative agents, such as Kathon CG and whitening agents, such as titanium dioxide.

According to a second aspect of the present invention there is provided a method of treatment or prophylaxis of traumatized or inflamed tissue surfaces which comprises administering to an animal in need of such treatment or prophylaxis a therapeutically effective amount of a vetinary composition as hereinbefore defined.

The vetinary composition is preferably applied topically to the traumatized or inflamed site.

The following illustrative examples relate to formulations made in accordance with the present invention and are intended to illustrate typical compositions. All materials utilized in the formulation are commercially available.

### EXAMPLE 1

| | |
|---|---|
| Ingredient | Percentage by weight |

Part I

| | |
|---|---|
| Sucrose mono/dipalmitate | 15.00 |
| Calcium stearate | 4.00 |
| Titanium dioxide | 0.08 |
| Preservative (Kathon CG) | 0.12 |
| Water | 67.40 |

Part II

| | |
|---|---|
| Mono/diglyceride of the edible fatty acids | 9.00 |
| Beeswax | 2.00 |
| Glycerol | 0.20 |
| Cetyl alcohol | 2.00 |
| dl α tocopherol | 0.20 |

In the preparation of Part I of the above formulation water (1101 g) was heated to 60° C. and titanium dioxide (1.2 g) and preservative Kathon CG (1.8 g) were added (alternatively a combination of citric acid and potassium sorbate or the parabenz may be used as a preservative) with stirring using a high shear mixer fitted with a disintegrating head. Sucrose mono/dipalmitate (225 g) was added slowly with vigorous stirring, maintaining the temperature at 600° C. When the dispersion was complete, calcium stearate (60 g) was then added slowly under the same conditions.

Once dispersion was complete, high shear mixing was stopped and mixing was continued using a combined propeller scrape blade system.

The components of Part II, mono/diglycerides (135 g), beeswax (30 g) and dl α tocopherol (3 g), were preheated to 60° C. and then added rapidly with vigorous stirring to Part I. The resulting mixture thickened rapidly and was packed while still at 50 to 60° C.

The veterinary composition produced is preferably applied topically to the traumatized or inflamed site, after the site has been cleaned and dried as much as possible. The composition should be applied liberally to form a thick surface coating, that should be left for approximately three days, before cleaning. If necessary a second application may then be made.

Initial trials have showed a definite improvement in all cases of traumatized or inflamed skin treated with the composition according to the present invention. In particular, dramatic improvements have been shown in the treatment of mud fever.

This composition has also been found to be effective in treating small domestic animals, in particular cats and dogs. For example, the use of this composition has been shown to clear up skin lesions and wet eczema in cats and skin infections in dogs. Also, application of the composition to a stiff and painful joint in a Labrador dog was found to relieve quickly the symptoms, and this composition promoted the healing of bruised and damaged pads on the paws of this animal.

This veterinary composition according to the present invention has also been found to have specific fungicidal properties, despite the fact that the composition itself requires the use of a preservative for such treatment, since the composition can itself be attached by fungi.

### EXAMPLE 2

| Ingredient | Percentage by weight |
|---|---|
| Sucrose mono/dipalmitate | 15.0 |
| Monopropylene glycol | 15.0 |
| Refined coconut oil | 4.0 |
| Glycerol | 1.0 |
| Ethoxylated sorbitan ester | — |
| Calcium stearate | 1.0 |
| Titanium dioxide | 0.2 |
| Kathon CG | 0.06 |
| Water | 63.74 |

The components were added to water (1019.8 g) at 60° C. with high shear mixing. Sucrose mono/dipalmitate (240 g) was added with stirring until an even dispersion was obtained, followed by calcium stearate (16 g), titanium dioxide (3.2 g) and Kathon CG (1 g) with stirring until a smooth even dispersion was obtained. Monopropylene glycol (240 g), refined coconut oil (64 g) and glycerol (16 g) were then added and the stirring continued for a further 5 minutes. On cooling, the material formed a soft thixotropic paste.

### EXAMPLE 3

| Ingredient | Percentage by weight |
|---|---|
| Sucrose mono/dipalmitate | 15.0 |
| Monopropylene glycol | 15.0 |
| Refined coconut oil | 4.0 |
| Glycerol | 1.0 |
| Ethoxylated sorbitan ester | — |
| Calcium stearate | 1.0 |
| Titanium dioxide | 0.2 |
| Kathon CG | 0.06 |
| Water | 63.74 |

The sequence of addition of components was the same as for Example 2. However, all mixing was carried out at ambient temperature. The resulting product was a thin fluid that required shaking before use to re-suspend the non-soluble components of the formulation.

### EXAMPLE 4

| Ingredient | Percentage by weight |
|---|---|
| Sucrose mono/dipalmitate | 15.0 |
| Monopropylene glycol | 12.0 |
| Refined coconut oil | 4.0 |
| Glycerol | 1.0 |
| Ethoxylated sorbitan ester | 2.0 |
| Calcium stearate | 1.0 |
| Titanium dioxide | 0.2 |
| Kathon CG | 0.06 |
| Water | 64.74 |

The components were added to water (1035.8 g) at 60° C. with high shear mixing. Sucrose mono/palmitate (240 g) was added and mixing continued until a smooth dispersion was obtained. Calcium stearate (16 g), titanium dioxide (3.2 g) and Kathon CG (1g) were then added. Mixing was continued until a smooth dispersion was obtained. Refined coconut oil (64 g), monopropylene glycol (192 g), glycerol (15 g) and ethoxylated sorbitan ester (32 g) were then added and stirring continued until a smooth dispersion was obtained. On cooling, a soft smooth cream was formed.

The veterinary composition of the present invention is biodegradable and, therefore, extremely environmentally friendly. Accordingly, the veterinary composition according to the present invention is easy and safe to use and, in particular, is ideal for use on organic farms.

It will also be understood that various alterations and modifications may be made to the above described veterinary composition without departing from the scope of the invention, and the present invention is not limited by the above examples. In particular, it is clear that the sucrose ester(s) used in the veterinary composition according to the present invention may be in any veterinarially acceptable carrier. The mixture of compositions chosen depending on the required consistency and feel of the desired final product. Furthermore, the veterinary composition according to the present invention may be used in combination with other medicaments or treatments, for example, the veterinary composition according to the present invention may be applied in combination with an insect repellent for both prevention and treatment of sweet itch. Also, it is foreseen that the veterinary composition according to the present invention may have applications for human use, in particular, for fungal infections such as ringworm, which may be passed from animal to human, by contact.

It is further believed that the veterinary composition according to the present invention may be useful in the treatment of other conditions, including use as an ear cleaning composition for relief from ear mites and malassezia. The composition according to the present invention may also prove useful in the treatment of inter-digital dermatitis and anal furunculosis.

1. The use of a vetinary composition comprising a sucrose ester or a mixture of sucrose esters for the manufacture of a medicament for the treatment or prophylaxis of traumatized or inflamed tissue surfaces.

2. The use according to claim 1, wherein said vetinary composition further comprises calcium or zinc stearate in a vetinarially acceptable carrier.

3. The use according to claim 2, wherein said vetinary composition contains a ratio of sucrose ester or mixture of sucrose esters to calcium or zinc stearate of between 2:1 and 6:1.

4. The use according to claim 3, wherein said vetinary composition contains a ratio of sucrose ester or mixture of sucrose esters to calcium or zinc stearate of around 3.75:1.

5. The use according to any preceding claim, wherein said sucrose ester or mixture of sucrose esters comprises 1 to 50% by weight of the total of said vetinary composition.

6. The use according to claim 5, wherein said sucrose ester or mixture of sucrose esters comprises 10 to 20% be weight of the total of said vetinary composition.

7. The use according to claim 6, wherein said sucrose ester or mixture of sucrose esters comprises 15% by weight of the total of said vetinary composition.

8. The use according to any preceding claim, wherein said sucrose ester or mixture of sucrose esters is within the range C₁₂-C₂₂.

9. The use according to any preceding claim, wherein said sucrose ester or mixture of sucrose esters comprises at least one unsaturated ester.

10. The use according to claim 9, wherein said at least one unsaturated ester is a fatty acid ester.

11. The use according to claim 10, wherein said mixture of sucrose esters comprises a mixture of sucrose monopalmitate and sucrose dipalmitate.

12. The use according to any preceding claim, wherein components of said vetinary composition are dissolved in at least one solvent selected from water, glycerol and cetyl alcohol.

13. The use according to any preceding claim, wherein said vetinary composition further comprises at least one additive selected from preservatives, emulsifying agents, whitening agents and additives that improve the “feel” of said composition when applied to skin or other tissue.

14. The use according to claim 13, wherein at least one of: a mixture of mono and diglycerides; lanolin; jojoba oil; and coconut oil is added to said vetinary composition to improve the “feel” thereof when applied to skin of other tissue.

15. The use according to any preceding claim, wherein said treatment or prophylaxis is of traumatized or inflamed tissue resulting from a fungal or microbial infection.

16. The use according to any of claims 1 to 14, wherein said treatment or prophylaxis is of traumatized or inflamed tissue resulting from an insect bite or sting.

17. The use according to any preceding claim, wherein said vetinary composition has an analgesic effect.

18. A method of treatment or prophylaxis of traumatized or inflamed tissue surfaces which comprises administering to an animal in need of such treatment or prophylaxis a therapeutically effective amount of a vetinary composition comprising a sucrose ester or mixture of sucrose esters.
